(19) United States
(12) Patent Application Publication
(10) Pub. No.: US 2007/0123935 A1
(43) Pub. Date: May 31, 2007

(54) Method and apparatus for contemporaneous formation of a body structure opening and homologous pedicle

(76) Inventor: Gene E. Myers, Sarasota, FL (US)

Correspondence Address: Mark J. Burns;Haugen Law Firm PLLP, Suite 1130, 121 South Eighth Street, Minneapolis, MN, 55402, US

(21) Appl. No.: 11/290,044

(22) Filed: Nov. 30, 2005

(51) Int. Cl. A61B 17/06

(52) U.S. Cl. 606/222

(57) ABSTRACT

The present invention provides a surgical needle apparatus having a piercing tip at a distal portion of a needle member. A first edge obliquely extends from the piercing tip to a heel portion of the needle member to define a partial boundary of a bore in fluid communication with a lumen of the needle member. A pair of cutting edges extend longitudinally from the first edge and extend radially outwardly above on outer surface of the needle member. The surgical needle apparatus is configured to create a body structure access site and an adjacent homologous pedicle for post-procedure sealing of the access site.

### FIELD OF THE INVENTION

The present invention relates generally to the field of apparatus and methods for creating and sealing of body structure access sites. More specifically, the present invention relates to an improved surgical needle apparatus and method for contemporaneously forming a body structure opening and homologous pedicle for closure thereof. The needle apparatus may be partially useful in accessing fluid- transporting structures such as vascular members.

### BACKGROUND OF THE PRIOR ART

Accessing the vascular system has, for several decades, been performed by one or more techniques of percutaneous entry. Early percutaneous entry involved puncturing of the site with a needle and then following the needle with an external hub and obturator. In this method, the needle punctured both the anterior vascular wall and the posterior vascular wall. The hub and obturator were blunt smooth surfaces that would displace and/or compact the vessel walls as they extended therethrough, as there was no removal of material during the procedure. Once positioned, the hub and obturator were progressively withdrawn until the needle drew blood, indicating proper placement in arterial lumen.

In such procedures, resealing of the arterial wall became difficult where the arterial wall had been stretched and/or deformed resulting in tissue necrosis and preventing proper recoil of the tissue.

More recently, a single wall cutting needle was introduced that only cuts into the anterior arterial wall, thus not requiring sealing of the posterior arterial wall. However, such needles and devices result in the removal of tissue from the arterial wall, including the vaso vasorum, leaving a hole in the tissue, once again making resealing of the site difficult.

The main disadvantage of this method is that resealing of the vessel wall requires external compression at the puncture site for a period of about forty-five minutes to one hour to allow closure by natural clotting. Following this, a patient must generally undergo 3-8 hours of bed rest to allow the seal to strengthen and usually should not return to normal activity for 2-3 days following the procedure. The medical, social and economic impact of this prolonged recovery period is substantial. Over 6 million such procedures occur annually in the United States, having an economic impact in the billions of dollars in terms of time and expense.

A number of vascular closure products have been introduced to address this problem. However, all such devices introduce non-homologous substances to the puncture site, such as bovine collagen. Even where most of such products perform under a wide range of conditions, none has been able to completely replace the procedure of manual compression. Specifically, for patients with larger puncture sites or patients taking anti-coagulant medications, compression remains the preferred means for sealing the puncture site. Sealing of a puncture site in a vascular wall, however, ultimately results in the formation of scar tissue that replaces a section of the vaso vasorum in the vessel wall. Accordingly, the puncture site remains a damaged area of the vessel wall subsequent to closure, and can act as a starting point to subsequent medical problems.

It would, therefore, be advantageous to provide an improved apparatus and method for overcoming these and additional shortcomings of the prior art.

More specifically, it would be advantageous to provide a method and apparatus for accessing a vascular system that minimizes damage to the arterial wall and vaso vasorum, and does not require removal of material therefrom, and further to provide a method and apparatus that does not require introduction of foreign materials to the vascular site for the sealing thereof.

It is a further object of the present invention to provide a method and apparatus for accessing a body structure in vivo that minimizes damage to such body structure and enables rapid re-sealment thereof.

### SUMMARY OF THE INVENTION

The present invention provides a surgical needle apparatus and method for contemporaneously creating a body structure access site and a homologous pedicle for sealing and closure thereof.

The surgical needle apparatus includes a needle member having a piercing tip at a distal portion thereof. The distal portion is configured to form a first edge extending obliquely to a proximal portion of the needle member. A bore is formed in the distal portion, about which bore the first edge extends defining opposing sides thereof and a pair of longitudinal cutting edges extend rearwardly from the first edge to define a recessed portion of the distal portion of the needle member. The bore is in fluid communication with the lumen of the needle member. The longitudinal cutting edges are configured to extend substantially parallel to an elongate axis defined by the needle member, such longitudinal cutting edges having a raised cutting surface extending radially from the outer surface of the needle member.

In operation, the piercing tip and first edge form a first incision in a body structure wall extending substantially perpendicular to the elongate axis of the needle member. The longitudinal cutting surfaces form incisions on opposing sides of the first incision, generally perpendicular thereto, to form an intact homologous pedicle in the body structure wall.

It is an object of the present invention to provide an apparatus and method for creating a vascular access site.

It is another object of the present invention to provide an apparatus and method for creating a homologous pedicle in an arterial wall for accessing and sealing of the vascular access site.

### BRIEF DESCRIPTION OF THE DRAWINGS

FIG. 1 is a top view of a prior art needle apparatus.

FIG. 2 is a top view of a needle apparatus in accordance with the present invention.

FIG. 3 is a cross-sectional view of the needle apparatus from FIG. 2 along cut line 3-3.

FIG. 4 is a cross-sectional view of the needle apparatus from FIG. 2 along cut line 4-4.

FIG. 5 is a cross-sectional view of the needle apparatus from FIG. 2 along cut line 5-5.

FIG. 6 is a cross-sectional view of the needle apparatus from FIG. 2 along cut line 6-6.

FIG. 7 is a side view of the needle apparatus of the present invention.

### DETAILED DESCRIPTION OF THE PREFERRED EMBODIMENTS

The objects and advantages enumerated above together with other objects, features, and advances represented by the present invention will now be presented in terms of detailed embodiments described with reference to the attached drawing figures which are intended to be representative of various possible configurations of the invention. Other embodiments and aspects of the invention are recognized as being within the grasp of those having ordinary skill in the art.

FIG. 1 shows a needle apparatus 100 from the prior art wherein a needle member 102 has a piercing tip 104 formed at a distal portion 106 thereof. The distal portion 106 is configured such that a top face 108 of the needle apparatus 100 obliquely extends rearwardly from the piercing tip 104 formed in the distal portion 106 thereof. The top face 108 of the needle apparatus 100 further defines a boundary of a bore 110 that is in fluid communication with the inner lumen 112 of the needle member 102. A proximal end 114 of the top face 108 of the needle apparatus 100 interfaces with the outer surface 116 of the needle member 102.

Top face 108 of needle apparatus 100 is further defined by first and second edges 109, 111 that diverge from piercing tip 104 and meet at heel portion 113 of proximal end 114 of top face 108. First and second edges 109, 111 form cutting surfaces to penetrate through, for example, the anterior wall of a vascular member. As distal portion 106 of needle apparatus 100 is pressed into the vascular member, heel portion 113 of first and second edges 109, 111 acts to cut and ultimately remove a section of the interior wall of the vascular member to leave an aperture in such vascular member. It is this aperture that induces the post-procedure healing difficulties described above.

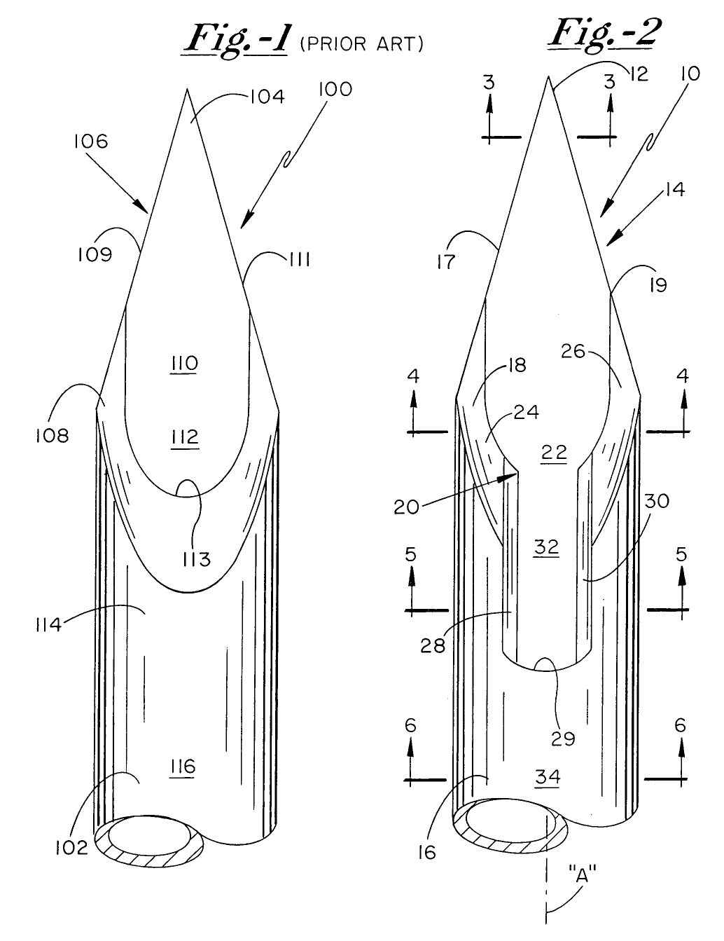

FIG. 2 shows a surgical needle apparatus 10 having a piercing tip 12 formed at a distal portion 14 of a needle member 16. In preferred embodiments, needle member 16 is substantially tubular in configuration, though variations of such a configuration are contemplated as being useful in apparatus 10 of the present invention. For example, needle member 16 may be fluted so as to provide for a desired entry characteristic into the vessel wall. The distal portion 14 of the needle member 16 further includes a first edge 17 obliquely extending proximally from the piercing tip 12 to a heel portion 20 of the surgical needle apparatus 10. A bore 22 is defined in the surgical needle apparatus 10 by opposing edges 17, 19, which diverge from piercing tip 12. A pair of longitudinal cutting edges 28, 30 extend rearwardly from heel portion 20, and define lateral boundaries of a recessed portion 32 that is coextensive with bore 22. As illustrated in FIG. 2, longitudinal cutting edges 28, 30 preferably terminate at end 29, which has a blunt or unsharpened surface so as not to function as a cutting surface.

First and second longitudinal cutting edges 28, 30 are generally shown extending parallel to an elongate axis “A” of the needle member 16. In a preferred aspect of the present invention, the first and second longitudinal cutting edges 28, 30 are configured to be raised so as to radially extend outwardly from an outer surface 34 of the needle member 16. Preferably, first and second longitudinal cutting edges 28, 30 extend radially outwardly from outer surface 34 of needle member 16 by between about 0.25 and 2 mm, and more preferably by between about 0.5 and 1 mm.

First and second longitudinal cutting edges 28, 30 preferably extend longitudinally from heel portion 20 to end 29 for a distance of between about 0.5 and 10 mm, and more preferably between about 1 and 7 mm. Moreover, first and second longitudinal cutting edges 28, 30 are preferably spaced apart by between about 0.5 and 5 mm, and more preferably between about 1 and 3 mm, such that recessed portion 32 has a width of between about 1 and 3 mm. While the dimensional specifications identified above provide a preferred construction of the present invention, it is contemplated that various dimensions may be utilized for various needle structures and for various applications that are outside the ranges set forth above. Such dimensional modifications, however, are contemplated as being included in the present invention. For example, first and second longitudinal cutting edges 28, 30 may extend in substantially non-parallel directions from heel portion 20. In some embodiments, first and second cutting edges 28, 30 may extend convergently from heel portion 20, while in other embodiments, first and second cutting edges 28, 30 may divergently extend from heel portion 20.

The raised aspect of longitudinal cutting edges 28, 30 act. to operably create thin incisions in the body structure wall and extending from opposed end portions of a first incision made by first and second edges 17, 19. In this manner, a homologous pedicle having a width of about 1-3 mm and a length of about 0.5-7 mm is created in the wall of the body structure being accessed by needle apparatus 10. The pedicle remains connected to the wall of the body structure, and provides a living homologous material that rapidly re-closes the access site once the needle apparatus 10 of the present invention is removed from the body structure.

The homologous pedicle has a shape defined by the relative orientation of first and second longitudinal cutting edges 28, 30. Accordingly, where first and second longitudinal cutting edges 28, 30 extend in substantially parallel relationship from heel portion 20, the homologous pedicle is a substantially three-sided flap that remains living and connected to the vessel member. Other configurations for the formed pedicle, however, are created through various relative configurations of first and second longitudinal cutting edges 28, 30.

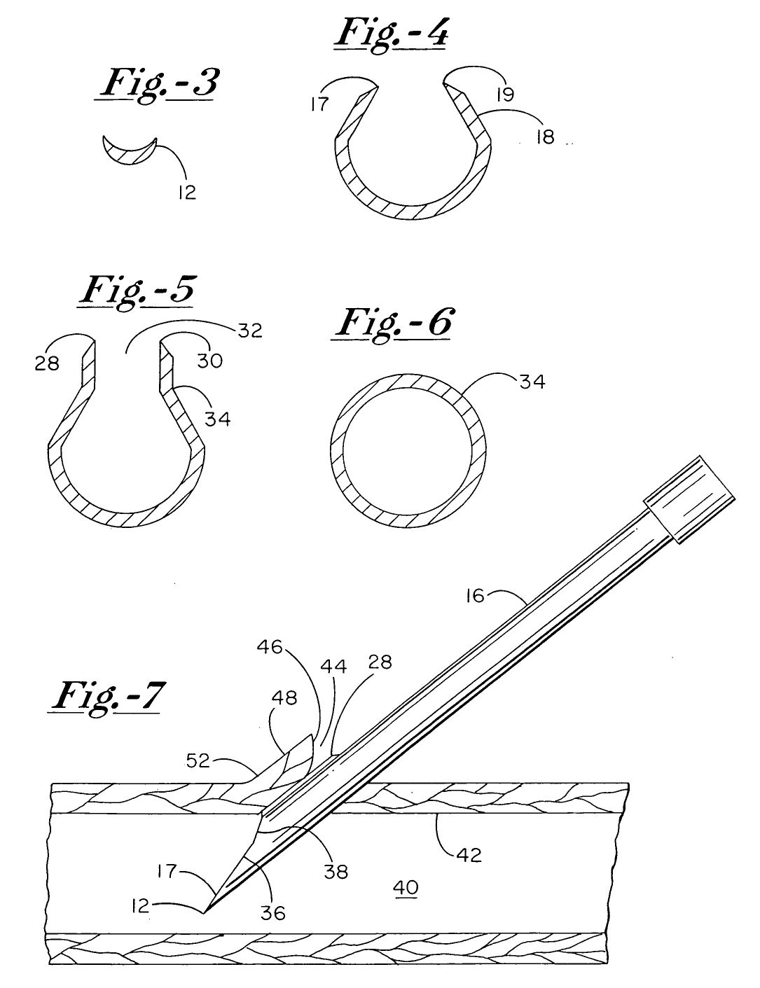

FIGS. 3-6 each provide a cross-sectional view of the needle apparatus 10 at different locations along the axis “A” defined by the elongate dimension of the needle member 16. FIG. 3 shows the generally radial cross section near the piercing tip 12 such that the opposing edges 17, 19 define terminating ends of the cross section shown at line 3-3. FIG. 4 shows a cross-section at distal portion 14 illustrating the beveled side profile 18 leading to first and second edges 17, 19.

FIG. 5 shows a cross-section of distal portion 14 including the longitudinal cutting edges 28, 30. Longitudinal cutting edges 28, 30 extend radially outwardly relative to the outer surface 34 of the needle member 16, as shown in comparison to the outer surface 34 of the needle member 16 in FIG. 6.

FIG. 7 shows an embodiment of the surgical needle apparatus 10 of the present invention placed within an arterial lumen 40. The piercing tip 12 allows percutaneous entry through the outer skin and tissue tract to the arterial wall 42 and creates an access site 44 therethrough. Surgical needle apparatus 10 may also be utilized non- percutaneously, such as during an open surgical procedure, to access a vessel such as arterial lumen 40. Other body structures, however, may be accessed by surgical needle apparatus 10 of the present invention, with examples of such alternative body structures including the synnovium, dura matter, pericardium, periturneam, and plura. Preferably, surgical needle apparatus 10 may be utilized with a variety of body structures to penetrate a wall thereof with minimal damage.

The piercing tip 12 and the first and second edges 17, 19 cooperate to create a first incision 46 in, for example, the arterial wall 42. The first incision 46 extends generally perpendicular to the axis “A” of the needle member 16. As the needle apparatus 10 is further advanced into the arterial lumen 40, the raised longitudinal cutting edges 28, 30 engage the arterial wall 42 at opposing end portions of the first incision 46 to simultaneously create second and third incisions 50, 52 and form a homologous pedicle 48. The second an third incisions 50, 52 preferably form a pair of generally parallel incisions that extend substantially perpendicular to the first incision 46 creating three sides of the homologous pedicle 48 in the arterial wall 42. The homologous pedicle 48 allows access to the arterial lumen 40 such that when the needle apparatus 10 is later removed from the arterial lumen 40, the homologous pedicle 48 will substantially return to its original position and orientation to provide a seal over the access site 44 and provide closure to the arterial wall 42.

As shown in the embodiment of FIG. 7, the beveled section 18 of the needle apparatus 10 may comprise a first beveled portion 36 and a second beveled portion 38, with each section 36, 38 being at a different oblique angle as it extends proximally with respect to the axis “A” of the needle member 16. Such an inflection relationship enables the preferred design of needle apparatus 10.

In use, the present invention provides a surgical needle apparatus 10 configured to contemporaneously access a body structure and create a homologous pedicle of the body structure wall. The present invention is further configured to cause minimal damage to the accessed body structure, such as by minimizing stretching of the tissue of the wall and/or minimizing the amount of material removed or otherwise damaged during such use. Instead, a homologous pedicle of body structure wall tissue is created for accessing the body structure which can be used to substantially reseal the body structure wall after completion of a procedure in which access is obtained. By way of example, apparatus 10 of the present invention may be particularly useful in preventing complications such as spinal fluid leak after a spinal tap, fluid/air leak after thoracentesis, blood/fluid/air leak from percutaneous needle lung biopsy, fluid/blood leak from pericardial centesis, fluid leak from arthrocentesis, and fluid leak following pericentesis.

Surgical needle apparatus 10 may be utilized in fluid-transporting vessels such as arteries, veins, and lymphatics in either an anti-grade or retrograde orientation. In applications requiring a retrograde insertion of surgical needle apparatus 10 into the fluid-transporting vessel, surgical needle apparatus 10 is preferably inserted into the anterior wall of the vessel in an orientation similar to that illustrated in FIG. 7. In particular, a retrograde application of surgical needle apparatus 10 is preferably conducted with first and second cutting edges 28, 30 being operably oriented facing substantially away from the vessel. The resultant pedicle 48 is formed such that fluid flow through the vessel contacts the connected portion of pedicle 48 prior to contacting the portion of pedicle 48 disconnected from the wall of the vessel. In such a manner, the extent to which homologous pedicle 48 is displaced from a pre-operative position co-extensive with the wall of the vessel minimized. Such minimization of displacement of pedicle 48 once surgical needle apparatus 10 is removed from the access site assists in the healing process.

In like manner to the above, applications requiring an anti-grade insertion of surgical needle apparatus 10 into the fluid-transporting vessel is preferably accomplished by inserting surgical needle apparatus 10 in an orientation such that first and second cutting edges 28, 30 are in facing relationship with the fluid-transporting vessel. This “upside-down” orientation of surgical needle apparatus 10 results in the formation of a homologous pedicle 48 having a flow contacting relationship with the fluid similar to that described above. Specifically, fluid flow preferably contacts a portion of pedicle 48 remaining connected to the vessel wall prior to contacting a portion of pedicle 48 disconnected from the vessel wall.

The embodiment of the present invention described herein is intended to be illustrative and not limiting as to the scope and spirit of the present invention.

What is claimed is:

1. A surgical needle comprising:
a) a needle member having a piercing tip formed at a distal end thereof;
b) a first edge obliquely extending proximally from said piercing tip to a heel portion of said needle member;
c) a second edge extending longitudinally from said first edge and having a cutting surface extending radially above an outer surface of said needle member.

2. The surgical needle according to claim 1 wherein said first edge has a first beveled section at a proximal portion thereof and a second beveled section at a distal portion thereof.

3. The surgical needle according to claim 1 wherein said second longitudinal edge is substantially parallel to an elongate axis of said needle member.

4. The surgical needle according to claim 1 wherein said first beveled edge has a cutting surface coextensive therewith.

5. A needle structure comprising:
a) a piercing tip formed at a distal end of a pair of converging beveled edge surfaces;
b) a pair of longitudinal cutting edges each extending proximally from said beveled edge surfaces to form opposing boundaries of a recess portion.

6. The needle structure from claim 5 wherein said beveled edge surfaces form opposing boundaries of a bore in said needle structure.

7. The needle structure from claim 5 wherein each of said beveled edge surfaces further comprise a raised cutting surface.

8. The needle structure from claim 5 wherein said pair of longitudinal cutting edge surfaces extend above a continuous outer surface of said needle structure.

9. A method for creating a body structure opening and a homologous pedicle, said method comprising the steps of:
a) forming a primary incision in said body structure by advancing a needle having a piercing tip and cutting surface into a wall of said body structure;
b) forming a pair of secondary incisions on opposing sides of said primary incision by further advancing said needle such that a corresponding pair of longitudinal cutting surfaces on said needle form said pair of secondary incisions in said wall of said body structure.

10. The method according to claim 9 wherein said pair of secondary incisions are substantially perpendicular to said first incision.

11. The method according to claim 9 wherein said body structure is a vascular member.

12. A surgical needle, comprising:
a) a cannula having a first open end including a piercing tip and a first cutting edge obliquely extending rearwardly to a heel portion of said first open end; and
b) a second cutting edge axially extending rearwardly from said heel portion and radially outwardly from an outer surface of said cannula.

13. A surgical needle as in claim 12 wherein said second cutting edge extends along said outer surface of said cannula.

14. A surgical needle as in claim 12, including a third cutting edge extending substantially parallel to said second cutting edge.

15. A surgical needle as in claim 14 wherein said second and third cutting edges define boundaries for a recessed portion of said first open end.

16. A surgical needle as in claim 12 wherein said second cutting edge extends radially outwardly from said outer surface of said cannula by between about 0.25 and 2 mm.

17. A method for creating an opening in a body structure and simultaneously forming a homologous pedicle of said body structure, the method comprising:
a) providing a surgical needle having:
(i) a cannula having a first open end including a piercing tip and a first cutting edge obliquely extending rearwardly to a heel portion of said first open end;
(ii) a second cutting edge axially extending rearwardly from said heel portion and radially outwardly from an outer surface of said cannula;
b) inserting said cannula into said body structure to an extent sufficient wherein said second cutting edge incises said body structure.

18. A method as in claim 17 wherein said body structure is a vascular member.

19. A method as in claim 17 wherein said second cutting edge extends along said outer surface of said cannula.

20. A method as in claim 17, including a third cutting edge extending along said outer surface of said cannula in spaced-apart relationship with said second cutting edge.

21. A method as in claim 17 wherein said cannula is inserted into said body structure in an orientation with said second cutting edge facing substantially away from said body structure.

22. A method as in claim 17 wherein said cannula is inserted into said body structure in an orientation with said second cutting edge in facing relationship with said body structure.
